(19) United States
(12) Patent Application Publication
(10) Pub. No.: US 2025/0000882 A1
(43) Pub. Date: Jan. 2, 2025

(54) LYSOPHOSPHATIDYLCHOLINE COMBINATIONS AS A THERAPY TO TREAT CARDIAC ARREST

(71) Applicant: THE FEINSTEIN INSTITUTES FOR MEDICAL RESEARCH, Manhasset, NY (US)

(72) Inventor: Junhwan KIM, New Hyde Park, NY (US)

(21) Appl. No.: 18/706,490

(22) PCT Filed: Nov. 4, 2022
(86) PCT No.: PCT/US2022/049003

§ 371 Date: May 1, 2024

Related U.S. Application Data
(60) Provisional application No. 63/276,131, filed on Nov. 5, 2021.

(51) Int. Cl. A61K 31/685; A61P 9/10

(52) U.S. Cl. CPC A61K 31/685; A61P 9/10

(57) ABSTRACT

The present invention provides for methods for reducing brain injury in or increasing survival of a subject that is suffering or has suffered cardiac arrest. These methods comprise administering an effective amount of a pharmaceutical composition comprising a lysophosphatidylcholine selected from the group consisting of lysophosphatidylcholine (18:1), lysophosphatidylcholine (18:2), lysophosphatidylcholine (22:6), and/or a combination thereof, optionally lysophosphatidylcholine (18:0), and a pharmaceutical carrier to the subject. The present invention further provides for the pharmaceutical compositions employed in these methods.

### CROSS-REFERENCE TO RELATED APPLICATIONS

This application claims the benefit of and priority to U.S. Provisional Application No. 63/276,131, filed Nov. 5, 2021, the disclosure of which is incorporated herein by reference in its entirety.

### FIELD OF INVENTION

This invention relates to new therapies and therapeutic compositions to assist in the recovery of subjects who have suffered cardiac arrest.

### BACKGROUND OF THE INVENTION

Cardiac arrest is a significant public health burden with few effective therapies presently available. Cardiac arrest is the abrupt loss of heart function, breathing and consciousness. The etiology of the condition usually results from a malfunction in the heart's electrical system (e.g., arrhythmias), which arrests the heart's pumping action, thereby stopping the flow of blood to the brain and other vital organs (e.g., brain, heart muscle, kidney, and liver) of the subject causing permanent damage to the tissues of these organs with death occurring within minutes. In addition to the loss of oxygen, the ceasing of circulation also stop the flow of non-oxygenated metabolites to these organs. Hence, cardiac arrest is often fatal unless appropriate steps, such as CPR and administering defibrillator shocks to the heart, are not taken immediately. Unfortunately, the restoration of blood to these tissues also causes damage to these tissues (i.e., ischemic-reperfusion injury). Disease and conditions which may cause electrical problems include ventricular fibrillation, coronary artery disease, physical stress, or structural changes to the heart.

Recent advances in the field of lipidomics have identified lipid metabolites as critical in many pathologic conditions (Küllenberg D, Taylor L A, Schneider M, et al: Health effects of dietary phospholipids. Lipids Health Dis 2012; 11:3). These lipid metabolites include phospholipids such as lysophosphatidylcholine (“LPC”) and sphinogomyelin, which are important intermediates in the synthesis and degradation of membrane phospholipids

LPC has the following structure:

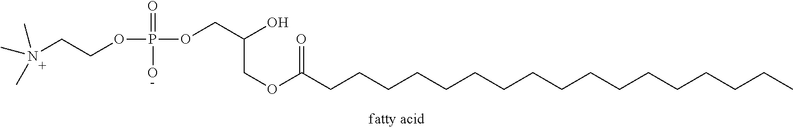

Seven major species of LPC have been identified in the plasma of mammals (including humans). These species are based on the identity of the fatty acid and are summarized in the below table:

| | | LPC |
|---|---|---|
| Fatty acid | Type of fatty acids | species |
| Palmitic acid | Saturated fatty acid | LPC(16:0) |
| Stearic acid | Saturated fatty acid | LPC(18:0) |
| Oleic acid | Monounsaturated fatty acid | LPC(18:1) |
| Linoleic acid | Polyunsaturated fatty acid (omega-6) | LPC(18:2) |
| Arachidonic acid | Polyunsaturated fatty acid (omega-6) | LPC(20:4) |
| Eicosapentaenoic acid | Polyunsaturated fatty acid (omega-3) | LPC(20:5) |
| Docosahexaenoic acid | Polyunsaturated fatty acid (omega-3) | LPC(22:6) |

US2020/0319190 A1 to J. Kim teaches that phospholipids, including LPCs, can be used as biomarkers for the identification and treatment of ovarian and pancreatic cancers. Further, it has been suggested that LPCs may be a potential therapy for treating brain ischemia or cognitive disorders. For example, Blondeau et. al. report that supplementing rats with LPC (14:0) provided protecting to neuronal cells after brain ischemia. (N. Blondeau et al., “A potent protective role of lysophospholipids against global cerebral ischemia and glutamate excitotoxicity in neuronal cultures”, J Cereb Blood Flow Metab, 2002. 22(7): p. 821-34). Further, Sugasini et al., indicate that supplementing the diet of rats with LPC (22:6) improved their cognitive functions. (D. Sugasini, et al., “Dietary docosahexaenoic acid (DHA) as lysophosphatidylcholine, but not as free acid, enriches brain DHA and improves memory in adult mice”, Sci. Rep., 2017. 7(1): p. 1126. However, until now, the role of LPCs in the setting of cardiac arrest and in ischemia-reperfusion injury has not been well studied.

There is a continuing need for novel therapies to treat subjects that are suffering or have suffered from cardia arrest. Based upon the discovery that decreased plasma LPC levels are associated with injury severity and outcomes in subjects suffering from cardiac arrest, the administration at least some specific LPC metabolites during or after the subject has suffered cardiac arrest may offer an innovative, non-oxygen-base therapy to treat these subjects by reducing brain injury and other vital organs, such as the heart and brain, and increasing the survival rate. These and other objectives are achieved by the present invention discussed below.

The citation or identification of any document in this application is not an admission that such document is available as prior art to the present invention

### SUMMARY OF THE INVENTION

The present invention provides for methods for reducing tissue injury (e.g. to cardiac muscle, kidney tissue, liver tissue, lung and brain tissue) and increasing the survival of a subject that is suffering or has suffered cardiac arrest. The inventive methods comprise administering an effective amount of a pharmaceutical composition comprising the specific, lipid metabolites lysophosphatidylcholine (18:1), lysophosphatidylcholine (18:2), lysophosphatidylcholine (22:6), herein referred to as LPC (18:1), LPC (18:2), LPC (22:6) respectively, or combinations thereof and optionally lysophosphatidylcholine (18:0), herein referred to as LPC (18:0), to the subject. Moreover, the present invention also provides for pharmaceutical compositions comprising a LPC selected from the group consisting of LPC (18:1), LPC (18:2), LPC (22:6), and/or a combination thereof, and optionally LPC (18:0), and a pharmaceutical carrier.

A method is provided for reducing brain injury in or increasing survival of a subject that is suffering or has suffered cardiac arrest, which comprises administering an effective amount of a pharmaceutical composition comprising a lysophosphatidylcholine selected from the group consisting of LPC (18:1), LPC (18:2), LPC (22:6), and/or a combination thereof, and a pharmaceutically acceptable carrier to said subject during or following cardiac arrest.

A method is provided for improving brain function in a subject that is suffering or has suffered cardiac arrest, which comprises administering an effect amount of a pharmaceutical composition comprising a lysophosphatidylcholine selected from the group consisting of LPC (18:1), LPC (18:2), LPC (22:6), and/or a combination thereof, and a pharmaceutically acceptable carrier to said subject during or following cardiac arrest.

Further provided is a method for reducing injury to heart muscle tissue, kidney tissue, liver tissue, lung tissue or any combination thereof in a patient that is suffering or has suffered cardiac arrest, which comprises administering an effect amount of a pharmaceutical composition comprising a lysophosphatidylcholine selected from the group consisting of LPC (18:1), LPC (18:2), LPC (22:6), and/or a combination thereof, and a pharmaceutically acceptable carrier to said subject during or following the cardiac arrest. In a specific embodiment, the tissue is heart muscle tissue. In yet another embodiment the injury is to lung tissue which may result in lung edema. An embodiment of the above-described methods and pharmaceutical compositions is where the pharmaceutical composition further comprises LPC (18:0).

In a further embodiment of the above-described methods and pharmaceutical compositions the pharmaceutical composition comprises LPC (18:1) and LPC (22:6) wherein the proportion of LPC (18:1), and LPC (22:6) is from about 0.1 to about 0.9. These pharmaceutical compositions may further comprise LPC (18:0), LPC (18:2), or both LPC (18:0) and LPC (18:2)

In another embodiment of the above methods and pharmaceutical compositions, the pharmaceutical composition further compromises at least one LPC that is selected from the group consisting of LPC (16:0), LPC (20:4), and LPC (20:5).

In an embodiment of the above methods the above-described pharmaceutical compositions are administered to the subject during cardiac arrest. In an alternative embodiment, the above-described pharmaceutical compositions are administered after the subject has suffered cardiac arrest, such as, for example within about 1 minute to about 120 minutes after the patient has suffered the cardiac event. In an alternative embodiment, the subject is administered the above-described pharmaceutical compositions within 30 minutes or within 10 to 20 minutes after the patient has suffered cardia arrest.

In yet another embodiment, kits containing pharmaceutical compositions comprising LPC (18:1), LPC (18:2), LPC (22:6), and/or a combination thereof, for treatment of cardiac arrest are provide. The kit may further comprise a pharmaceutical composition comprising other LPCs such as, for example, LPC (18:0). Such kits contain materials useful for the treatment of cardiac arrest as described herein.

In yet another embodiment, kits containing pharmaceutical compositions comprising LPC (18:1) and LPC (22:6) and/or a combination thereof or pharmaceutical combinations comprising LPC (18:1) and LPC (22:6) and/or a combination thereof and LPC (18:0), LPC (18:2), or both LPC (18:0) and LPC (18:2) for treatment of cardiac arrest are provide. The kit may further comprise a pharmaceutical composition comprising other LPCs such as, for example, LPC (18:0). Such kits contain materials useful for the treatment of cardiac arrest as described herein.

It is noted that in this disclosure and particularly in the claims and/or paragraphs, terms such as “comprises”, “comprised”, “comprising”, and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by the following Detailed Description.

### BRIEF DESCRIPTION OF THE DRAWINGS

FIG. 1 illustrates the comparison of plasma LPC levels between survivors (n=9) and nonsurvivors (n=27) with hospital discharge was performed using the Mann-Whitney U test.

FIG. 2A-F illustrates the comparison of the levels of individual plasma lysophosphatidylcholine (LPC) species in cardiac arrest patients. FIG. 2A-F, cardiac arrest patients who survived to discharge (n=9) have significantly higher individual LPC levels in plasma than patients who died in the hospital (n=27) except for LPC (20:4). Data are presented as mean±SE of the mean. The Mann-Whitney U test was used for the comparison.

FIG. 3A-G illustrates post-resuscitation levels of plasma lysophosphatidylcholine (LPC) after 10- or 14-min cardiac arrest. Changes in the levels of (FIG. 3A) total LPC and (FIG. 3B-G) individual LPC species until 2 hr of post resuscitation after 10- or 14-min cardiac arrest (n=6 in each group). The y-axis is percentage of LPC levels compared with the baseline levels, and the x-axis is time after cardiopulmonary resuscitation following cardiac arrest. Data are presented as mean±SEM. The difference between the two groups was compared using the Mann-Whitney U test. * represents the significance of changes in total and individual LPC species after resuscitation compared with their baselines in 10- or 14-min group using the Jonckheere trend test.

FIG. 4A-D demonstrates the beneficial effect of lysophosphatidylcholine (LPC) supplementation on rat survival and neurologic function after 10 min of cardiac arrest (CA). (FIG. 4A), Comparing survival rates between the LPC (18:1) and vehicle groups for 72 hr after cardiopulmonary resuscitation (n=12 for each group). (FIG. 4B), Modified neurologic deficit score (mNDS) at 72 hr after resuscitation for surviving rats in both groups. (FIG. 4C), Comparing survival rates between the LPC (22:6), LPC(18:0), and vehicle groups for 72 hr after resuscitation (n=12 for each group). (FIG. 4D), mNDS at 72 hr after resuscitation for surviving rats in the three groups. Differences between the groups were compared using the Mann-Whitney U test.

FIG. 5A and FIG. 5B demonstrate results from brain histological examination after lysophosphatidylcholine (LPC) administration after cardiac arrest. (FIG. 5A) The number of neurons in cresyl violet-stained sections per 200-μm linear length of hippocampal CA1 pyramidal layers was counted in rats survived for 72 hr in the vehicle (n=9), LPC (18:1) (n=8), LPC(18:0) (n=5), and LPC(22:6) (n=9) and compared with sham rats(n=4). (FIG. 5B), The number of FJB-positive cells in the medial part of the cerebral cortex (0.16 mm2) was counted in rats survived for 72 hr in the vehicle (n=9), LPC (18:1) (n=8), LPC (18:0) (n=5), and LPC (22:6) (n=9) groups and compared with the sham animals (n=4). The difference was compared using the Steel-Dwass test with the vehicle group as the reference.

FIG. 6 illustrates the effect on survival by depleted LPC species LPC (18:0), LPC (18:1), LPC (22:6) using the 12 minutes of asphyxia-induced cardiac arrest rat model.

FIG. 7 illustrates on survival by the combination of LPC (18:0), LPC (18:1) and LPC (22:6) (“the LPC Combination”) using the 12 minutes of asphyxia-induced cardiac arrest rat model.

FIG. 8 depicts the plasma troponin I level for the individual LPC species LPC (18:0), LPC (18:1), LPC (22:6) using the 12 minutes of asphyxia-induced cardiac arrest rat model.

FIG. 9 depicts the plasma troponin I level for the LPC Combination using the 12 minutes of asphyxia-induced cardiac arrest rat model.

FIG. 10 depicts the appearance and the time to SSEP N10 peak after 12 minutes of cardiac arrest for the individual LPC species, LPC (18:1) and the LCP Combination.

FIG. 11 depicts Kaplan-meier survival analysis showing that LPC (18:2) treatment improved rat survival after 10 min cardiac arrest.

FIG. 12 depicts brain function assessed with modified neurological deficit scores (mNDS) at 48 hours and 72 hours after cardiac arrest between vehicle and LPC (18:2) treated rats after 10 min cardiac arrest.

FIG. 13 depicts that ejection fraction at 2 h after resuscitation was significantly decreased in vehicle treated rats but not in LPC (18:2) treated rats after 10 min cardiac arrest, indicating LPC (18:2) treatment improved heart function compared to vehicle treatment at 2 h after resuscitation.

FIG. 14 depicts decreased lung wet-to-dry weight indicates reduced lung edema by LPC (18:2) treatment after cardiac arrest.

FIG. 15A and FIG. 15B depict that supplementing LPC (18:1) decreased inflammatory IL-5 levels and increase anti-inflammatory IL-10 levels in rat plasma using the 10 minutes of asphyxia-induced cardiac arrest rat model.

The following detailed description, given by way of example, but not intended to limit the invention solely to the specific embodiments described, may best be understood in conjunction with the accompanying drawings.

### DETAILED DESCRIPTION OF THE INVENTION

This invention provides for reducing brain injury in or increasing survival of a subject that is suffering or has suffered cardiac arrest, which comprises administering an effective amount of a pharmaceutical composition comprising a LPC selected from the group consisting of LPC (18:1), LPC (18:2), LPC (22:6), and/or a combination thereof, and a pharmaceutically acceptable carrier to said subject during or following cardiac arrest.

This invention further provides for a method for improving brain function in a subject that is suffering or has suffered cardiac arrest, which comprises administering an effect amount of a pharmaceutical composition comprising a LPC selected from the group consisting of LPC (18:1), LPC (18:2), LPC (22:6), and/or a combination thereof, and a pharmaceutically acceptable carrier to said subject during or following cardiac arrest.

This invention also provides for a method for reducing injury to heart muscle tissue, kidney tissue, lung, liver tissue or any combination thereof in a patient that is suffering or has suffered cardiac arrest, which comprises administering an effect amount of a pharmaceutical composition comprising a LPC selected from the group consisting of LPC (18:1), LPC (18:2), LPC (22:6), and/or a combination thereof, and a pharmaceutically acceptable carrier to said subject during or following the cardiac arrest. In an embodiment, the tissue in heart muscle tissue. In another embodiment, the tissue is lung tissue.

Further, this invention provides for a method for increasing anti-inflammatory IL-10 levels and decreasing inflammatory IL-6 levels in plasma in a subject that has suffered cardiac arrest, which comprises administering an effect amount of a pharmaceutical composition comprising a LPC selected from the group consisting of LPC (18:1), LPC (18:2), LPC (22:6), and/or a combination thereof, and a pharmaceutically acceptable carrier to said subject.

This invention provides for a pharmaceutical composition for reducing brain injury or increasing survival in a patient that is suffering or has suffered from cardiac arrest which comprises an effective amount of a comprising a LPC selected from the group consisting of LPC (18:1), LPC (18:2), LPC (22:6), and/or a combination thereof, and a pharmaceutically acceptable carrier.

This invention provides for a pharmaceutical composition for reducing lung edema injury or increasing survival in a patient that is suffering or has suffered from cardiac arrest which comprises an effective amount of a comprising a LPC selected from the group consisting of LPC (18:1), LPC (18:2), LPC (22:6), and/or a combination thereof, and a pharmaceutically acceptable carrier. In a specific embodiment, a pharmaceutical composition comprising LPC (18:2) is administered to reduce lung edema.

An embodiment of the above-described methods and pharmaceutical compositions is where the pharmaceutical composition further comprises LPC (18:0).

In a further embodiment of the above-described methods and pharmaceutical compositions wherein the proportion of LPC (18:1), and LPC (22:6) is from about 0.1 to about 0.9.

In another embodiment of the above methods and pharmaceutical compositions, the pharmaceutical composition further compromises at least one LPC that is selected from the group consisting of LPC (16:0), LPC (20:4), and LPC (20:5).

In another embodiment of the above methods and pharmaceutical compositions, the pharmaceutical composition comprises lysophosphatidylcholine (18:1), lysophosphatidylcholine (18.2), and lysophosphatidylcholine (22:6).

In another embodiment of the above methods and pharmaceutical compositions the pharmaceutical composition comprises lysophosphatidylcholine (18:1), lysophosphatidylcholine (22:6), and lysophosphatidylcholine (18:0).

In yet another embodiment of the above methods and pharmaceutical compositions, the pharmaceutical composition comprises the pharmaceutical composition comprises LPC (18:1) and LPC (22:6).

In an embodiment of the above methods the above-described pharmaceutical compositions are administered to the subject during cardiac arrest. In an alternative embodiment, the above-described pharmaceutical compositions are administered after the subject has suffered cardiac arrest, such as, for example within about 1 minute to about 120 minutes after the patient has suffered the cardiac event. In an alternative embodiment, the subject is administered the above-described pharmaceutical compositions within 30 minutes or within 10 to 20 minutes after the patient has suffered cardiac arrest.

In some of the above embodiments the subject is a human. In another embodiment, the subject is a non-human animal, such as for example, mammals, such as felines (e.g., cats), canines (e.g., dogs), equines (e.g., horses, donkeys, or zebras), llamas, pigs, and bovines, and birds (e.g., chickens).

In some of the above methods, tissues in the body of the subject experiencing cardiac arrest are protected from damage (i.e., a reduction in injury to tissue in a treated subject compared to a non-treated subject is observed). In some embodiments, the tissue is cardiac muscle tissue. In other embodiments, the tissue is brain tissue. In yet other embodiments, the tissue is kidney tissue. In yet other embodiments, the tissue is liver tissue. In yet other embodiments, the tissue is lung tissue.

In one embodiment, the pharmaceutical composition comprises LPC (18:1), LPC (18:2), LPC (22:6), LPC (18:0), and a pharmaceutically acceptable carrier. Examples of acceptable pharmaceutically acceptable carriers are well known in the art and typically include, but are not limited to, additive solution-3 (AS-3), saline, phosphate buffered saline, Ringer's solution, lactated Ringer's solution, Locke-Ringer's solution, Krebs Ringer's solution, Hartmann's balanced saline solution, and heparinized sodium citrate acid dextrose solution.

Pharmaceutical compositions of embodiments comprise a therapeutically effective amount of one or more LPC (18:1), LPC (18:2), LPC (22:6), LPC (18:0) dissolved or dispersed in a pharmaceutically acceptable carrier. The term “therapeutically effective amount” as used herein has the standard meaning known in the art to mean an amount sufficient to treat a subject afflicted with a condition or disease (e.g., cardiac arrest) or to halt the progression of the condition or disease or alleviate a symptom or a complication associated with the condition or disease. The exact dose will be ascertainable by one skilled in the art using known techniques (e.g., Ansel et al., Pharmaceutical Dosage Forms and Drug Delivery; Lieberman, Pharmaceutical Dosage Forms (vols. 1-3, 1992), Dekker, ISBN 0824770846, 082476918X, 0824712692, 0824716981; Lloyd, The Art, Science and Technology of Pharmaceutical Compounding (1999); and Pickar, Dosage Calculations (1999)).

The preparation of a pharmaceutical composition that contains one or more LPC (18:1), LPC (18:2), LPC (22:6), LPC (18:0), or any additional LPC, and optionally an additional active ingredient will be known to those of skill in the art in light of the present disclosure, as exemplified by Remington's Pharmaceutical Sciences, 18th Ed. Mack Printing Company, 1990. For human administration, it will be understood that preparations should meet sterility, pyrogenicity, general safety and purity standards as required by FDA Office of Biological Standards or corresponding authorities in other countries. Preferred compositions are lyophilized formulations or aqueous solutions. As used herein, “pharmaceutically acceptable carrier” includes any and all solvents, buffers, dispersion media, micelles, surfactants, antioxidants, preservatives (e.g. antibacterial agents, antifungal agents), isotonic agents, absorption delaying agents, salts, preservatives, antioxidants, proteins (e.g., albumin), drugs, drug stabilizers, excipients, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

The pharmaceutical compositions of the present invention being administered by any conventional means, typically the inventive compositions are administered intravenously or intramuscularly. In certain embodiments, the method further comprises administering to the individual a therapeutically effective amount of at least one or more additional therapeutic agent, e.g. an agent that is typically used to treat cardiac arrest.

For the treatment of cardiac arrest, the appropriate dosage of the one or more LPC (18:1), LPC (18:2), LPC (22:6), LPC (18:0), and/or combinations thereof (when used alone or in combination with one or more other additional therapeutic agents) will depend on the route of administration, the body weight of the patient, the severity and course of the disease, whether the pharmaceutical compositions are administered for preventive or therapeutic purposes, previous or concurrent therapeutic interventions, the patient's clinical history and previous response to LPC (18:1), LPC (18:2), LPC (22:6), and/or LPC (18:0) treatment, and the discretion of the attending physician. The practitioner responsible for administration will, in any event, determine the concentration of active ingredient(s) in a composition and appropriate dose(s) for the individual subject. Various dosing schedules including but not limited to single or multiple administrations over various time-points.

One typical dosage would be in the range from about 1 μg/kg body weight to 1000 mg/kg body weight. In other non-limiting examples, a dose may also comprise from about 1 μg/kg body weight, about 5 μg/kg body weight, about 10 μg/kg body weight, about 50 μg/kg body weight, about 100 μg/kg body weight, about 200 μg/kg body weight, about 350 μg/kg body weight, about 500 μg/kg body weight, about 1 mg/kg body weight, about 5 mg/kg body weight, about 10 mg/kg body weight, about 50 mg/kg body weight, about 100 mg/kg body weight, about 200 mg/kg body weight, about 350 mg/kg body weight, about 500 mg/kg body weight, to about 1000 mg/kg body weight or more per administration, and any range derivable therein.

The LPC (18:1), LPC (18:2), LPC (22:6), and/or LPC (18:0) of certain embodiments will generally be used in an amount effective to achieve the intended purpose. For use to treat cardiac arrest and symptoms resulting from said cardiac arrest the LPCs of these embodiments, or pharmaceutical compositions thereof, are administered or applied in a therapeutically effective amount. Determination of a therapeutically effective amount is well within the capabilities of those skilled in the art, especially considering the detailed disclosure provided herein.

For systemic administration, a therapeutically effective dose can be estimated initially from in vitro assays, such as cell culture assays. A dose can then be formulated in animal models to achieve a circulating concentration range that includes the IC₅₀ as determined in cell culture. Such information can be used to determine useful doses more accurately in humans. Initial dosages can also be estimated from in vivo data, e.g., animal models, using techniques that are well known in the art. One having ordinary skill in the art could readily optimize administration to humans based on animal data. Those of ordinary skill in the art will readily optimize effective dosages and administration regimens as determined by good medical practice and the clinical condition of the individual patient.

The attending physician for patients treated with the LPC (18:1), LPC (18:2), LPC (22:6), and/or LPC (18:0) of certain embodiments would know how and when to terminate, interrupt, or adjust administration due to toxicity, organ dysfunction, and the like. Conversely, the attending physician would also know to adjust treatment to higher levels if the clinical response were not adequate (precluding toxicity). The magnitude of an administered dose in the management of the cardiac arrest will vary with the severity of the condition, with the route of administration, and the like. The severity of the condition may, for example, be evaluated, in part, by standard prognostic evaluation methods. Further, the dose and perhaps dose frequency will also vary according to the age, body weight, and response of the individual patient.

The LPC (18:1), LPC (18:2), LPC (22:6), and/or LPC (18:0) described herein may be administered in combination with one or more other treatments or agents or “therapeutic agents” for use in treatment of cardiac arrest. The term “therapeutic agent” encompasses any treatment or agent administered to treat cardiac arrest in an individual in need of such treatment. Such treatments include, for example, cardiopulmonary resuscitation (CPR), therapeutic hypothermia, extracorporeal membrane oxygenation, and defibrillation, which may be applied to a subject in cardiac arrest. Such additional therapeutic agent may comprise any active ingredients suitable for treatment of cardiac arrest, preferably those with complementary activities that do not adversely affect each other. Such agents may include, for example, idocaine, epinephrine, atropine, naloxone, and vasopressin to name a few.

In another aspect, an article of manufacture (e.g., a kit) containing materials useful for the treatment of cardiac arrest as described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle).

The label or package insert indicates that the composition is used for treating the condition of choice, e.g. cardiac arrest. The article of manufacture may comprise (a) a first container with a composition contained therein, wherein the composition comprises LPC (18:1), LPC (18:2), LPC (22:6), and/or LPC (18:0).

Kits in certain embodiments may further comprise a package insert indicating that the compositions can be used to treat a particular condition. Alternatively, or additionally, the kit may further comprise a second (or third) container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

“And/or” as used herein, for example, with option A and/or option B, encompasses the separate embodiments of (i) option A, (ii) option B, and (iii) option A plus option B.

Where a numerical range is provided herein, it is understood that all numerical subsets of that range, and all the individual integers contained therein, are provided as part of the invention.

All combinations of the various elements described herein, including all subsets, are within the scope of the invention unless otherwise indicated herein or otherwise clearly contradicted by context.

The present invention will be further illustrated in the following Examples, which are given for illustration purposes only and are not intended to limit the invention in any way.

### Example 1

The data and the conclusions reported herein are found in abstract by Nishikimi, M., et al.,” Identification of Decreased Plasma Lysophosphatidylcholine Using Phospholipidomics in Cardiac Arrest: A Novel Therapeutic Application” online published Nov. 9, 2020 (www.ahajournals.org/doi/10.1161/circ.142.suppl_4.155), herein incorporated by reference, and a publication by Nishikimi, M., et al., “Phospholipid Screening Postcardiac Arrest Detects Decreased Plasma Lysophosphatidylcholine: Supplementation as a New Therapeutic Approach”, Crit. Care Med., online published Jul. 2, 2021, herein incorporated by reference including all documents cited therein.

### Methods and Materials

### Human Plasma Sample Collection

Patients' information was obtained retrospectively from the electronic medical charts. Blood samples were obtained from 36 post-cardiac arrest patients. Blood of cardiac arrest patients was drawn within 1 hour after return of spontaneous circulation (ROSC). Plasma was separated within 1 hour of blood draw by centrifugation at 1,000×g for 10 minutes and stored at −80° C. For the control group, blood was collected from 25 people including both healthy volunteers and non-cardiac arrest patients of comparable age and gender.

### Animal Preparation

The study was conducted using adult male Sprague-Dawley rats (400-500 g, Charles River Laboratories, Wilmington, MA) utilizing procedures for asphyxia-induced cardiac arrest and cardiopulmonary resuscitation (CPR). Briefly, asphyxial cardiac arrest was induced by stopping ventilation after injecting vecuronium bromide (2 mg/kg). After 10 or 14 minutes of asphyxia-cardiac arrest, ventilation was restarted with chest compressions. Typically, rats achieve ROSC within 1 minute after the initiation of CPR. Rats were extubated 2 hours after ROSC. Blood samples were collected from the femoral artery at baseline and multiple time points during cardiac arrest or postresuscitation. Plasma was separated using centrifugation at 1,000×g for 10 minute and stored at −80° C. Brain tissue was harvested from animals that survived for 72 hours after perfusion with saline and fixed with 4% paraformaldehyde.

### Plasma LPC Supplementation

Over 90% of plasma LPC in humans and rats contains palmitic acid (16:0), stearic acid (18:0), oleic acid (18:1), linoleic acid (18:2), arachidonic acid (20:4), or docosahexaenoic acid (22:6). These species are noted as LPC (16:0), LPC (18:0), LPC (18:1), LPC (18:2), LPC (20:4), and LPC (22:6), respectively.

To test the beneficial effect of LPC supplementation, LPC (18:1) was first administered because it was the only commercially available species out of the four LPC species decreased after 10 minute of cardiac arrest. The study was then extended to test the effect of attached fatty acid by including custom-made LPC (22:6) and LPC (18:0). Different groups of control rats for each set of experiment for accurate assessment were used. Rats were randomized into each group (n=12). Baseline characteristics of rats were not different between the groups in each set of experiment.

The LPC species mixed with 0.5% bovine serum albumin (BSA) in phosphate-buffered saline (0.5 mL) was injected over 1 minute via the femoral vein cannula beginning 1 minute after ROSC, with vehicle group injected with only BSA. 6 mg/kg of LPC was injected, because this dosage preserved LPC level in post-resuscitation plasma for 2 hours after 10-minute cardiac arrest. The purities of the injected LPC species were over 98%.

Survival was followed for 72 hours with brain function evaluated in a blinded manner using the modified neurologic deficit score (mNDS) after resuscitation for surviving rats. The mNDS is expressed as percentage, with 100% representing perfect score.

### Analysis of Plasma Phospholipids and Brain Morphology

Plasma phospholipids were analyzed using plasma samples obtained from humans and rats following our established high-performance liquid chromatography-mass spectrometry (HPLC-MS) method. For brain histology, serial coronal sections (14 μm) were prepared from the fixed brain tissue using a cryostat (CM1900, Leica, Germany). The sections were used for Nissl staining to determine the number of surviving neurons in hippocampal CA1 region or Fluoro-Jade B (FJB) staining to count the number of degenerative neurons in the cortex.

### Phospholipid Screening

To identify phospholipids that are associated with cardiac arrest, we performed the least absolute shrinkage and selection operator (LASSO) logistic regression analysis.

### Statistical Analysis

Kaplan-Meier curves compared survival between the LPC and vehicle groups using the Wilcoxon test. For phospholipids levels, Mann-Whitney U test was used to compare two independent groups. Repeated measures analysis of variance was used to compare the vital signs between two groups. Spearman correlation coefficients were calculated to evaluate the correlation between blood pH and plasma LPC levels. For changes in plasma LPC levels postreperfusion, we performed Jonckheere-Terpstra test. Statistical analyses were performed using the JMP software (Version 14.2, SAS Institute, Cary, NC). p values of less than 0.05 were considered statistically significant. Data were expressed as the mean±se of the mean.

### Results

Plasma LPC Levels Correlate with Injury Severity and Outcomes after Cardiac Arrest in Humans and Rats.

As described herein, it has been observed in humans that plasma LPC is significantly decreased after cardiac arrest. Furthermore, plasma LPC levels inversely correlate with the duration of cardiac arrest and survival outcome of patients. Moreover, administering LPC (18:1) and LPC (22:6), but not LPC (18:0) improved brain function and survival in rats after 10 minutes of asphyxia-induced cardiac arrest. After 12 minutes of more severe cardiac arrest, administering all three LPC species together as a combination improved rat brain function and survival.

Phospholipidomics were performed on plasma samples from cardiac arrest patients and controls from North Shore University Hospital. A phospholipid survey on human cardiac arrest and control plasma was performed. Using mass spectrometry analysis followed by multivariable regression analyses, it was demonstrated that plasma LPC levels were an independent discriminator of cardiac arrest. Furthermore, the levels correlated with the duration from arrest until return of spontaneous circulation (ROSC), which in turn correlates with injury severity. Consistently, the plasma LPC levels were associated with survival outcome of patients.

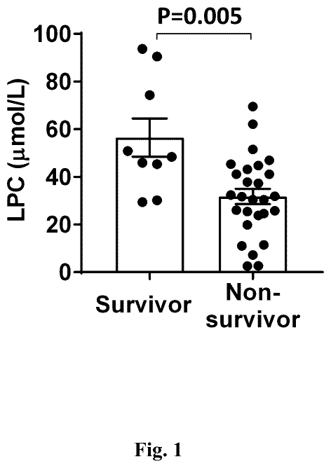

The same trend was observed in the rat model where rats after 14 min cardiac arrest had significantly lower plasma LPC levels than rats after 10 min cardiac arrest (FIG. 1). Further, LPC levels began to decrease continuously following resuscitation, but not during the ischemic phase of cardiac arrest. Overall, the data indicates a significant association between plasma levels of LPC and injury severity and outcomes after cardiac arrest in humans and rats. The data also suggests that decreased plasma LPC is a major contributor to mortality and brain damage postcardiac arrest.

### Phospholipid Survey in Human Cardiac Arrest Patients

Plasma phospholipids levels were compared between 36 cardiac arrest patients (median age of 74 yr; 16 males [44%]) and 25 controls (median age of 72 yr; 12 males [48%]). Among 36 post-cardiac arrest patients, nine patients survived to discharge and 27 patients died in the hospital.

HPLC-MS analysis found seven phospholipids, LPC, phosphatidylcholine, phosphatidyletha-nolamine, sphingomyelin, phosphatidylinositol, lysophosphatidylinositol, and lysophosphatidyl-ethanolamine, from human plasma. The levels of LPC, sphingomyelin, phosphatidylinositol, lysophosphatidylinositol, and lysophosphatidylethanolamine were significantly decreased in cardiac arrest patients with phosphatidylethanolamine showing a decreased trend. However, phosphatidylcholine levels were not different between the groups.

Out of the seven phospholipids, the LASSO logistic regression analysis identified lysophosphatidylinositol and LPC as the best discriminators between human cardiac arrest and control (λ. 1SE=0.076. Using these species, a multivariable logistic regression analysis identified LPC as an only independent variable that is associated with cardiac arrest with an odds ratio of 0.71 (95% CI, 0.56-0.90; p=0.005). The odd ratio of LPI was 0.55 (95% CI, 0.23-1.30; p=0.18).

It was also found that the LPC levels correlated with injury severity of patients. The LPC level was higher in patients who survived to discharge than those who died in the hospital (56.5 vs 31.8 μM, p=0.005). Furthermore, the LPC level was correlated with the time from emergency call until ROSC (rs=0.55, p<0.001) and blood pH of cardiac arrest patients (rs=0.40, p=0.018). Overall, the data from phospholipid survey show a significant association between the diminished plasma LPC levels and poor out-comes after cardiac arrest.

### Changes in Individual LPC Species in Human Cardiac Arrest Patients

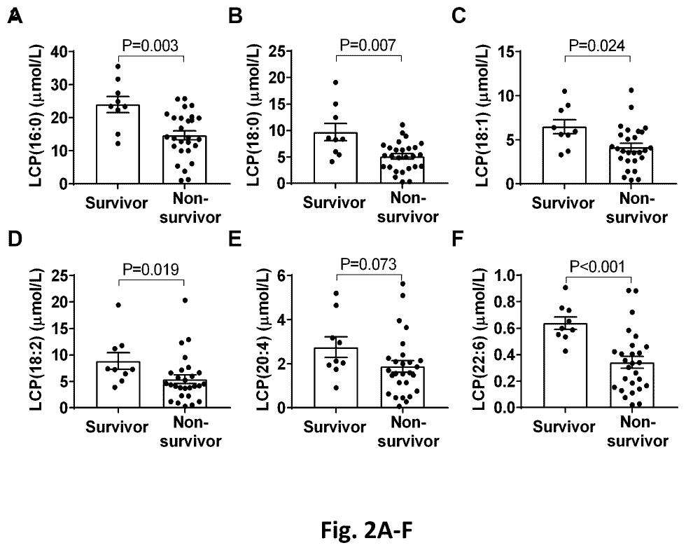

Changes in individual LPC species were then examined. It was found that the levels of all six LPC species were significantly lower in the cardiac arrest group than the control group by 57-80%. Furthermore, the levels of individual LPC species were significantly lower in the cardiac arrest patients who died in the hospital than those who survived to discharge by approximately 40%, except for LPC (20:4), which still showed a decreasing trend (FIG. 2 A-F). Although there were some variations depending on the attached fatty acids, all major LPC species showed a similar decrease pattern.

### LPC Decrease in Postresuscitation Plasma in Rats

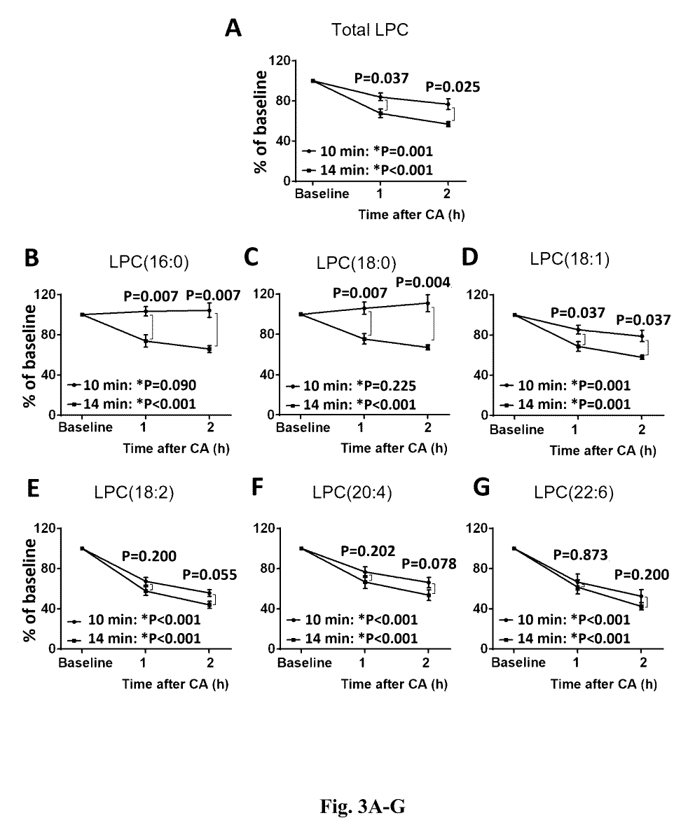

In order to confirm the relationship between LPC decrease and injury severity found in humans with the rat model, LPC levels were compared in postresuscitation rat plasma after 10-minute cardiac arrest (less severe injury) or 14-minute cardiac arrest (more severe injury). It was found that the total LPC content was significantly decreased in both groups, with greater decrease in the 14-minute group (FIG. 3A). The levels of LPC (16:0) and LPC (18:0) did not decrease until 2 hours following 10-minute cardiac arrest, but significantly decreased following 14-minute CA; comparison between the two groups showed a significant difference of these species (FIGS. 3, B and C). The levels of LPC (18:1) were decreased in both 10- and 14-minute cardiac arrest groups, and there still was a significant difference in the decrease between the groups (FIG. 3D). The levels of LPC (18:2), LPC (20:4), and LPC (22:6) were significantly decreased in both groups, and there was a trend for a greater decrease in the 14-minute group (FIG. 3 E-G). Overall, the association between LPC levels and injury severity found in human cardiac arrest was also observed in our rat cardiac arrest models.

### LPC Changes During Ischemic Phase of Cardiac Arrest

To test if LPC decrease begins during the ischemic phase that continues into resuscitation or is a function of solely resuscitation, LPC levels were measured in plasma obtained during various durations of ischemia. It was found that no LPC species decreased until 30 minutes of cardiac arrest, indicating that the decrease in LPC primarily begins after resuscitation but not during the ischemic phase of CA.

### Therapeutic Effect of LPC Supplementation

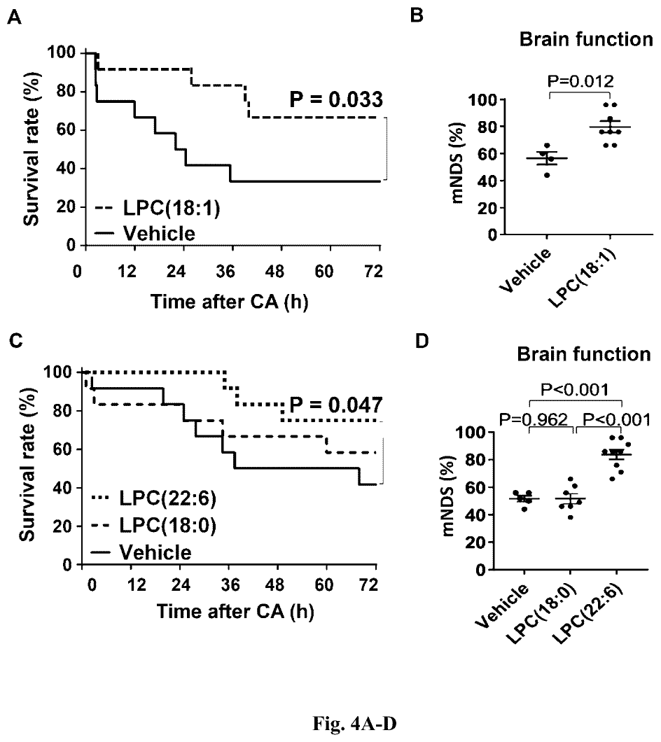

As discussed above, the beneficial effects of LPC (18:1) supplementation were tested after 10-minute cardiac arrest. The Kaplan-Meier survival curve shows that LPC (18:1) administration significantly improved rat survival (FIG. 4A). It was also found that surviving rats treated with LPC (18:1) had significantly higher mNDS scores than the vehicle group at both 24 hours (66% vs 42%) (not shown) and 72 hours (76% vs 58%) (FIG. 4B). After observing improved outcomes with LPC (18:1), custom-made LPC (22:6) were tested in order to determine the beneficial effect of different fatty acids and LPC (18:0) was tested to determine whether undepleted species also exert a beneficial effect. Interestingly, supplementation of only LPC (22:6) improved rat survival as well as neurologic outcomes at both 24 hours (51% vs 40%) (not shown) and 72 hours (86% vs 52%) (FIGS. 4, C and D).

However, mean arterial pressure or heart rate were not changed with supplementation of any of the LPC species for 90 minutes after resuscitation, suggesting no improved blood flow contributing to improved survival. Furthermore, no improved outcomes were observed with LPC (18:0), demonstrating that supplementation of only depleted LPC species is beneficial.

Brain Histology with LPC Supplementation

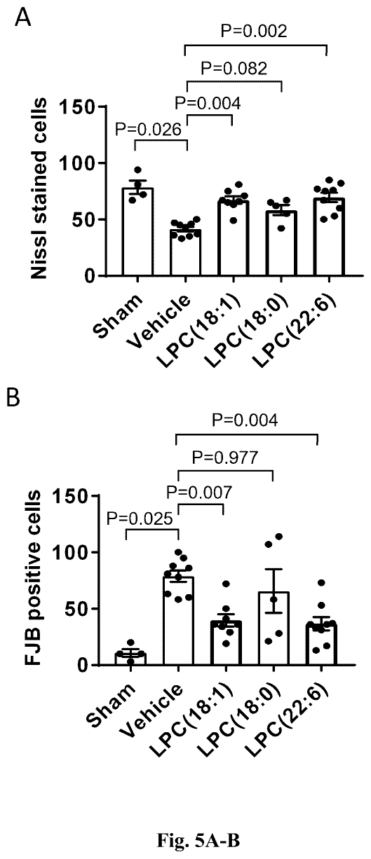

Improved cellular morphology was found with LPC supplementation at 72 hours post-resuscitation. Nissl staining showed that the number of neurons in the hippocampus was 51%, 86%, and 88% of control values in the vehicle, LPC (18:1), and LPC (22:6) groups, respectively (FIG. 5A). Additionally, neuronal shrinkage, a common morphological alteration after ischemia, was also reduced in the LPC groups. FIB staining also showed that degenerating cells in the cortex, which was increased by seven-fold in the vehicle group compared with the control value, was decreased to a three-fold increase with LPC (18:1) and LPC (22:6) supplementation (FIG. 5B). Again, LPC (18:0) supplementation showed a minimal effect on neuronal cellular morphology.

Administering a Combination of LPC (18:1), and LPC (22:6), and LPC (18:0) is More Effective than Administering the Individual Species

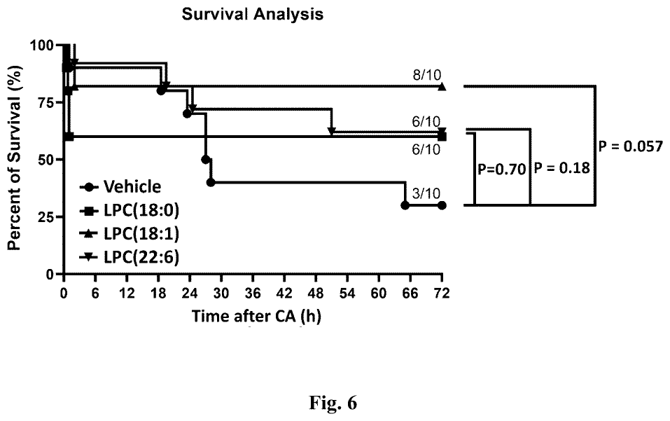

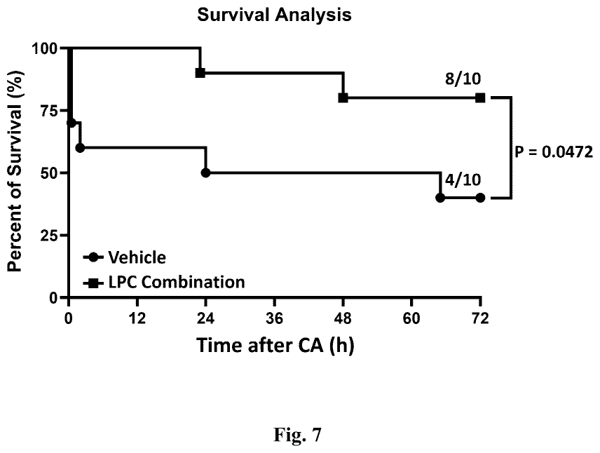

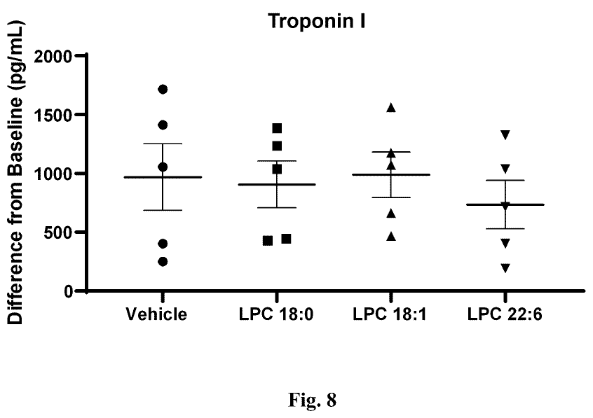

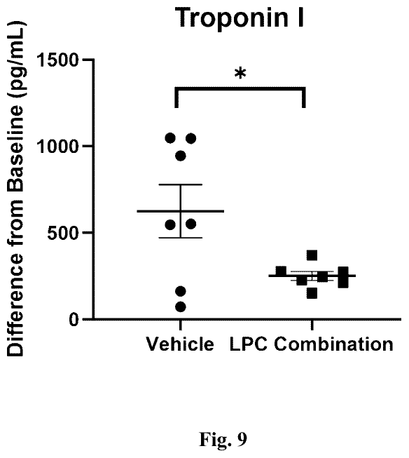

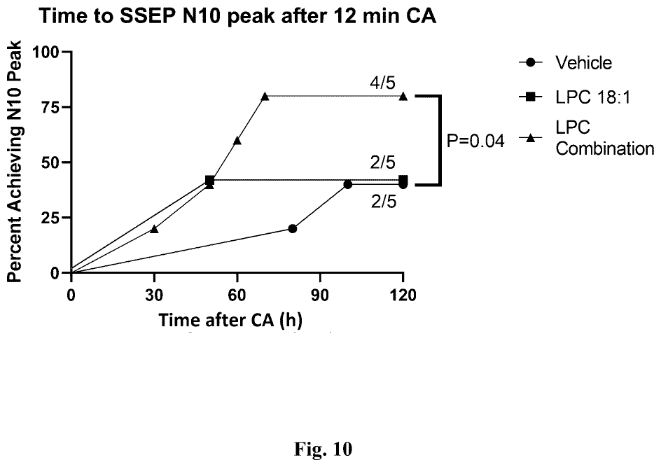

The therapeutic effects of LPC species as well as combination of all three species was then tested using 12 minutes of cardiac arrest, which induces more severe injury than 10 minutes of cardiac arrest. The Kaplan-Meier survival curve shows that LPC (18:1) administration most significantly improved rat survival (FIG. 6). The combination also showed significantly improved rats' survival (FIG. 7). Also compared was the plasma troponin I level to assess the effects of individual LPC species LPC (18:0), LPC (18:1), LPC (22:6) and the LPC Combination for heart function (see FIG. 8 and FIG. 9 respectively). Surprisingly, only the LPC Combination showed a decrease in plasma troponin I level. Since these rats are severely injured and mNDS are not appropriate to measure their brain function, we used SSEP to compare the brain function between LPC (18:1) and LPC combination groups. The data indicate that while the LPC Combination facilitates the return of the SSEP signal, the did not happen with the individual LPC species (FIG. 10). Taken together the data indicated that the LPC combination is surprisingly more effective that the individual species in protecting the brain and the heart after cardiac arrest.

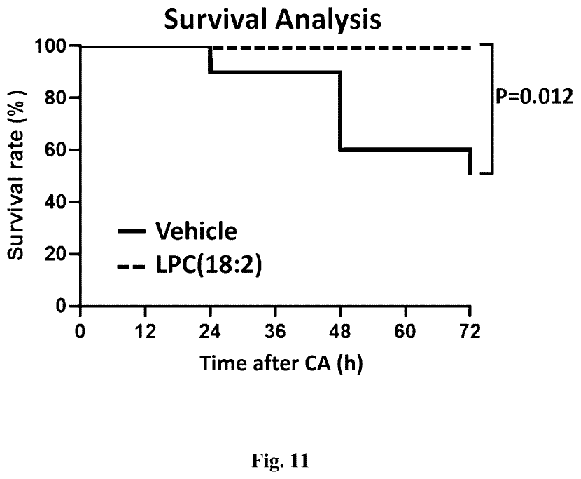

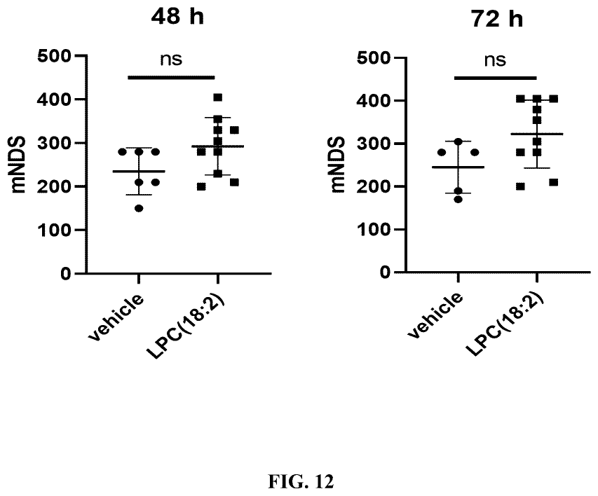

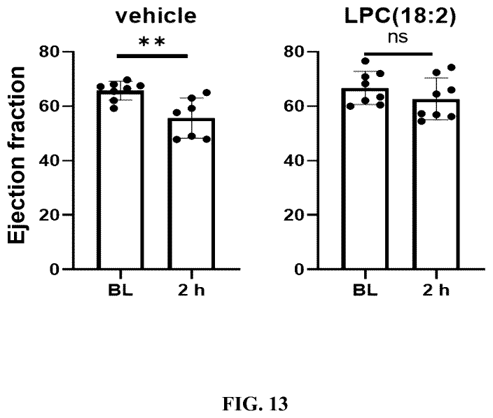

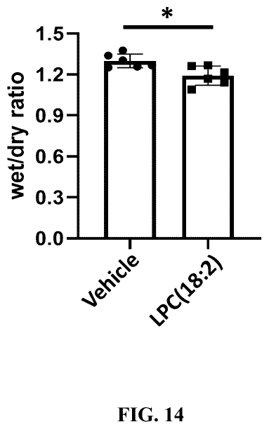

The beneficial effects of LPC (18:2), LPC(20:4), and LPC (16:0) for their beneficial effects using the same cardiac arrest model were also tested. It was found that that only LPC (18:2) significantly improve survival of the three LPC species tested (FIG. 11). The neuroprotective effect of LPC (18:2) was not as good as LPC (18:1) or LPC (22:6) (FIG. 12). Furthermore, none of the LPC (18:2) treated rats were able to walk whereas 40 to 50% of LPC (22:6) and LPC (18:1) treated rats were able to walk. Instead, it was found that LPC (18:2) improved heart ejection fraction at 2 h after resuscitation (FIG. 13), indicating that LPC (18:2) protects the heart. Reduced lung wet-to-dry ratio was also found with LPC (18:2) supplementation (FIG. 14), which suggests reduced lung edema. Overall, these data indicate that LPC (18:2) protects organs other than the brain. Therefore, combination of LPC (18:2) with LPC (18:1) and/or LPC (22:6) may be more effective than supplementing individual LPC species.

### Administrating LPC (18:1) Decreased IL-6 and Increased IL-10

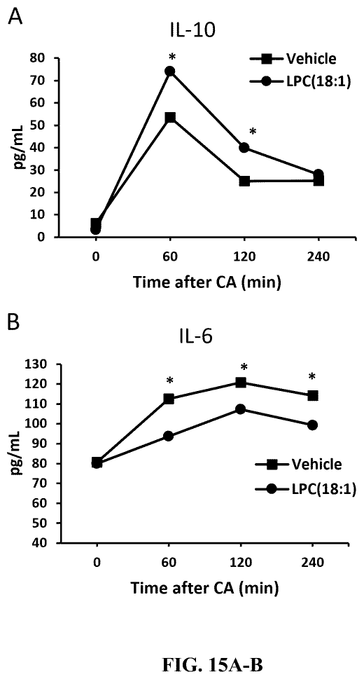

The beneficial effects of LPC administration on inflammatory IL-6 and anti-inflammatory IL-10 levels in blood plasma after the subject suffered cardiac arrest was also demonstrated. Using the 10 minutes of asphyxia-induced cardiac arrest rat model, supplementing LPC (18:1) decreased inflammatory IL-6′ levels and increased the anti-inflammatory IL-10 levels in rat plasma after 10 minutes of cardiac arrest (FIG. 15A and FIG. 15B).

Having thus described in detail preferred embodiments of the present invention, it is to be understood that the invention defined above is not limited to particular details set forth in the above description as many apparent variations thereof are possible without departing from the spirit or scope of the present invention. All publications cited herein and publications referenced in those documents (including manufacturer's instructions, descriptions and product specifications), are expressly incorporated herein by reference in their entirety and may be employed in the practice of the invention.

1-41. (canceled)

42. A method for treatment of a subject that is suffering, or has suffered cardiac arrest, which comprises administering an effective amount of a pharmaceutical composition comprising a lysophosphatidylcholine selected from the group consisting of lysophosphatidylcholine (18:1), lysophosphatidylcholine (18.2), lysophosphatidylcholine (22:6), and/or a combination thereof, and a pharmaceutically acceptable carrier to said subject during or following cardiac arrest.

43. The method according to claim 42, wherein the pharmaceutical composition further comprises at least one lysophosphatidylcholine that is selected from the group consisting of lysophosphatidylcholine (16:0), lysophosphatidylcholine (20:4), lysophosphatidylcholine (20:5) and lysophosphatidylcholine (18:0).

44. The method according to claim 42, wherein the pharmaceutical composition is administered to said subject during cardiac arrest.

45. The method according to claim 42, wherein the pharmaceutical composition is administered to said subject within 1 min to 120 min after suffering cardiac arrest.

46. The method according to claim 42, wherein the pharmaceutical composition is administered to said subject within 30 minutes after suffering cardiac arrest.

47. The method according to claim 42, wherein the pharmaceutical composition comprises lysophosphatidylcholine (18:1), lysophosphatidylcholine (18.2), and lysophosphatidylcholine (22:6).

48. The method according to claim 43, wherein the pharmaceutical composition comprises lysophosphatidylcholine (18:1), lysophosphatidylcholine (22:6), and lysophosphatidylcholine (18:0).

49. The method of claim 42, wherein said method of treatment is administered for (i) reducing brain injury in the subject; (ii) improving brain function in the subject; (iii) increasing survival of the subject; (iv) reducing injury to heart muscle tissue, kidney tissue, liver tissue, or any combination thereof in the subject; and/or (v) reducing plasma IL-10 levels and decreasing IL-6 levels in the subject.

50. The method according to claim 43, wherein the pharmaceutical composition is administered to said subject during cardiac arrest.

51. The method according to claim 43, wherein the pharmaceutical composition is administered to said subject within 1 min to 120 min after suffering cardiac arrest.

52. The method according to claim 51, wherein the pharmaceutical composition is administered to said subject within 30 minutes after suffering cardiac arrest.

53. The method of claim 49, where the injury is to heart muscle tissue.

54. The method of claim 49, where the injury is to lung tissue.

55. A pharmaceutical composition for treatment of a patient that is suffering, or has suffered from cardiac arrest, which comprises an effective amount of a comprising a lysophosphatidylcholine selected from the group consisting of lysophosphatidylcholine (18:1), lysophosphatidylcholine (18.2), lysophosphatidylcholine (22:6), and/or a combination thereof, and a pharmaceutically acceptable carrier.

56. The pharmaceutical composition according to claim 55, wherein said composition further comprises at least one lysophosphatidylcholine that is selected from the group consisting of lysophosphatidylcholine (16:0), lysophosphatidylcholine (20:4), lysophosphatidylcholine (20:5) and lysophosphatidylcholine (18:0).

57. The pharmaceutical composition according to claim 55, which comprises lysophosphatidylcholine (18:1), lysophosphatidylcholine (18.2), and lysophosphatidylcholine (22:6).

58. The pharmaceutical composition according to claim 56, which comprises lysophosphatidylcholine (18:1), lysophosphatidylcholine (22:6), and lysophosphatidylcholine (18:0).

59. A kit comprising a pharmaceutical composition comprising one or more of lysophosphatidylcholine (18:1), lysophosphatidylcholine (18.2), lysophosphatidylcholine (22:6), and/or lysophosphatidylcholine (18:0).

60. The kit of claim 59, wherein the pharmaceutical composition further comprises at least one lysophosphatidylcholine that is selected from the group consisting of lysophosphatidylcholine (16:0), lysophosphatidylcholine (20:4), lysophosphatidylcholine (20:5) and lysophosphatidylcholine (18:0).

61. The kit of claim 59, wherein the pharmaceutical composition comprises lysophosphatidylcholine (18:1), lysophosphatidylcholine (18.2), and lysophosphatidylcholine (22:6).

62. The kit of claim 60, wherein the pharmaceutical composition comprises lysophosphatidylcholine (18:1), lysophosphatidylcholine (22:6), and lysophosphatidylcholine (18:0).
